(19) United States
(12) Patent Application Publication
(10) Pub. No.: US 2004/0219605 A1
(43) Pub. Date: Nov. 4, 2004

(54) Fluid planar lipid layer-based membrane-anchored ligand system with defined ligand valency and methods of use thereof

(76) Inventors: Terri H. Finkel, Wynnewood, PA (US); Zhengyu Ma, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/820,262

(22) Filed: Apr. 8, 2004

Related U.S. Application Data
(60) Provisional application No. 60/461,223, filed on Apr. 8, 2003.

(51) Int. Cl. G01N 33/53; C12N 1/00

(52) U.S. Cl. 435/7.1; 435/317.1

(57) ABSTRACT

Fluid planar lipid layer-based membrane-anchored ligand systems with defined ligand valency and methods of use thereof are provided.

This application claims priority to U.S. Provisional Application 60/461,223 filed Apr. 8, 2003, the entire disclosure of which is incorporated by reference herein.

### FIELD OF THE INVENTION

The present invention relates to the fields of cellular and molecular biology. More specifically, the invention provides materials and methods which facilitate the determination of the valency requirement of receptors for membrane-anchored ligands and observation of the spatial distribution and interaction parameters of ligand-receptor interactions at the interface of membrane-membrane contacts in real time.

### BACKGROUND OF THE INVENTION

Several publications and patent documents are cited throughout the specification in order to describe the state of the art to which this invention pertains. Each citation is incorporated herein as though set forth in full.

Interactions between membrane anchored ligands and receptors are common and essential biological events. For example, the critical event of T lymphocyte (T cell) activation is a result of membrane-membrane contact between T cells and antigen presenting cells. A variety of ligand-receptor interactions take place between the two opposing membranes, including, major histocompatibility complex (MHC)-peptide and T cell receptor (TCR), LFA-1 and ICAM-1, CD2 and CD48, as well as B7, CD80, or CTLA-4 and CD28.

Systems employing artificial membranes incorporating membrane-anchored ligands have been previously established as tools for studying cell-cell interactions (Watts, T. H. et al. (1984) Proc Natl Acad Sci USA. 81(23): 7564-7568; Grakoui, A. et al. (1999) Science. 285(5425): 221-227). There are drawbacks, however, to these currently available systems. In the approach reported by Watts et al, the whole transmembrane ligand is incorporated into liposomes first and the liposomes are then put on a glass surface to form the lipid bilayer. Because of the interactions between the intracellular domains of the ligands and the hydrophilic glass surface, the ligands cannot diffuse freely in the bilayer.

In the approach described by Grakoui et al, the extracellular domain of a ligand with a GPI anchor is expressed in mammalian cells, the cells are lysed with detergent and the ligands are purified with affinity chromatography. The GPI-anchored ligands are then incorporated into the lipid bilayer as described by Watts et al. The GPI anchor ensures the fluidity of the ligands. However, when expressed in mammalian cells, GPI-anchored proteins tend to be enriched in lipid rafts, which is detergent insoluble. Therefore, the product of the affinity purification of the ligands from the cell lysate is likely to comprise clusters of GPI-anchored ligands in lipid rafts. Moreover, other cell surface proteins that are enriched in lipid rafts may be co-purified with the GPI-anchored ligands and subsequently incorporated into the lipid bilayer. Additionally, the valency of the ligands in the lipid layer cannot be determined. Grakoui et al. also describe directly labeling the ligand which can have detrimental effects on the ability of the ligand to interact with its receptor.

The instant invention provides superior artificial membrane systems which overcome these drawbacks and enable the skilled person to determine the valency requirement of receptors for membrane-anchored ligands and observe the spatial distribution of ligand-receptor interactions at the interface of membrane-membrane contacts in real time.

### SUMMARY OF THE INVENTION

In accordance with the instant invention, a method for producing a fluid planar lipid layer-based membrane-anchored ligand system with defined ligand valency is provided. An exemplary method entails contacting a solid surface with a lipid layer containing lipids conjugated to a first specific binding pair member; functionally linking a ligand to a second specific binding pair member which has binding affinity for said first binding pair member, the second member comprising at least one binding site for binding said first member; and contacting the lipid layer with the linked ligand whereby contact of the lipid layer with said second binding pair member functionally linked to said ligand results in anchoring of the ligand to said lipid, thereby forming a fluid planar lipid layer-based membrane-anchored ligand system with defined ligand valency. In one aspect, the ligand is functionally linked to the second binding pair member through binding interaction with the first binding pair member.

In yet another embodiment of the invention, a fluid planar lipid layer-based membrane-anchored ligand system produced by the forgoing method is provided. The method facilitates studies of monovalent and multivalent membrane interactions. For example, monovalent reactions can be studied using nickel and histidine tags as binding pair members. Multivalent interactions can be studied, for example, using biotin and streptavidin as binding pair members.

A variety of different ligands and their interactions can be assessed using the methods of the present invention. Such ligands include without limitation, I-EK-MCC and I-AK-CA, neuropilin-1, LFA1, DC-SIGN, ICAM1, ICAM3, MHC, TCR, CD100, SEMA4A, CD40, CD40L, CD80, CD86, CD28, SEMA7A, CD72, TIM2, B7-H1/B7-DC, B7-1/B7-2, B7RP-1, B7H3, 4-1BBL, CD27L, OX40L, OX40, CD27, 4-1BB, ICOS, CTLA4, PD1,plexin-C1,CD4, CD8, CKR family members, CXCR4, CCR5, CCR3, gamma-cytokine receptor family members, IL2R, IL4R, IL7R, IL15R, SRA, CD68, LOX1, HSP receptors, CD91, TLR4, TLR2, CD36, CD40, CD14, v3 integrin, and TNFR family members, TNFR, FAS, and FASL.

Suitable surfaces useful in the method of the invention include glass coverslips, biacore chips, sensor chips and tissue culture plates. Specific binding member pairs encompassed within the present invention include nickel-histidine, biotin -streptavidin, antibody-antigen, lectin-carbohydrate, and complementary oligonucleotides.

The methods of the invention also include analysis of cell-receptor interactions and thus in one aspect, the method includes T cells, antigen presenting cells, macrophages, B cells, neurons, a fibroblast, an endothelial cell, an epithelial cell, a synoviocyte, a muscle cell, a stem cell, and dendritic cells. Additionally, membrane-virus interactions may be assessed. In a preferrred embodiment, the virus is HIV.

In yet another embodiment, the methods of the invention include analyzing test compounds for the ability to disrupt the membrane interactions under study.

Finally, the invention includes a kit for practicing the instantly disclosed methods. An exemplary kit includes lipids, a solid surface, a plurality of first and second binding members; and optionally at least one ligand of interest. The ligand or the first and second binding member pairs are optionally detectably labeled.

### BRIEF DESCRIPTION OF THE DRAWINGS

FIG. 1 is flow chart of a process of generating a fluid planar lipid layer-based membrane-anchored ligand system with defined ligand valency exemplified by the use of streptavidin.

FIG. 2 is a schematic drawing of the planar lipid bilayer. The specific binding pairs are exemplified with streptavidin/biotin and HisTag/Ni-NTA and the ligands are exemplified by MHC-pep and ICAM-1.

FIG. 3 is a schematic drawing of the planar lipid bilayer on a Dextran-cushioned coverglass.

FIG. 4 is a schematic drawing of various models of the T-cell/APC interaction.

FIG. 5A is a Coomassie Blue stained gel of ICAM-1 purified from a Ni2+ column. FIG. 5B is a chart depicting the dose response adhesion of fluorescently-labeled D10.IL2 cells to 96 well ELISA plates coated with purified ICAM-1. The fluorescence of the plates (CFSE (carboxyfluorescein succinimidyl ester) reading) is graphed in comparison to the concentration of ICAM-1.

FIG. 6 is a graph depicting the adhesion of CFSE labeled D10.IL2 cells to POPC monolayers containing DOPE-bio or DOPC and contacted with SA-(ICAM-1-bio)₂.

FIGS. 7A-7D are images of resting D10.IL2 cells fixed with 2.5% formaldehyde and incubated with a mixture of KJ16 (rat) and F23.1 (mouse) (FIG. 7A), D10.IL2 cells with TCR/CD3 crosslinked with anti-CD3e antibody (2C11) and goat anti-hamster antibody (FIG. 7B), resting T cells stained with a mixture of anti-TCR Vβ8.2 antibody F23.1 and anti-CD3ε antibody 500A2 (hamster), then stained with corresponding non-crossreacting secondary antibodies labeled with Cy3 (for CD3ε) or Cy5 (for TCR) (FIG. 7C), and resting T cells stained with a mixture of anti-CD3ε antibody 500A2 and anti-LFA-1 antibody I21/7 (rat), then stained with corresponding non-crossreacting secondary antibodies labeled with Cy3 (for LFA-1) or Cy5 (for CD3ε) (FIG. 7D). The intensity of the marked pixels, calculated by the Slidebook™ software (Intelligent Imaging Innovations, Inc; Denver, Colo.) was plotted on the graphs by the images. FIG. 7E is a graph of the % FRET for FIGS. 7A-7D. Data are representative of 8 cells for each group.

FIGS. 8A and 8B are fluorescent images of a Dextran-cushioned POPC:POPC/10% DOPE-bio bilayer and a POPC:POPC bilayer, respectively, bound with streptavidin-FITC. FIG. 8C is a fluorescent image of a Dextran-cushioned POPC:POPC/10% DOPE-bio bilayer incubated with streptavidin-FITC that was pre-blocked with free biotin prior to incubation with the lipid bilayer. The bar represents 10 μm.

FIG. 9 provides fluorescence (FITC) and DIC (Differential Interference Contrast) imaging of T cells at the lipid bilayer surface.

FIGS. 10A and 10B represent the results from the functional assays of I-Ek-MCC with wild-type and null peptides.

FIG. 11 is a graph of the ICAM-1-AviTag-HisTag functional assay.

FIGS. 12A and 12B are graphs of the results from T cell calcium flux assays employing streptavidin-I-Ek multimers anchored on a POPC/POPC/DOPE-bio bilayer in media containing 10% FBS (FIG. 12A) and induced by CH27 cells pulsed with PCC in medium containing 10% FBS (FIG. 12B).

FIGS. 13A-13C are images of lipid bilayers comprising POPC and DOPE-NDB (FIG. 13A), POPC/DOPE-bio and bound by SA-FITC (FIG. 13B), or POPC/DOPE-1% NTA-Ni bilayer and bound by 6×His tagged ICAM-1-FITC (FIG. 13C) that were photobleached over an initial area with a diameter of 30 μm and monitored over time.

FIGS. 14A-14C are images demonstrating cellular migration over time on glass (FIG. 14A), a lipid bilayer comprised of POPC/1% DOPE-NBD/1% DOPE-bio (FIG. 14B), or lipid bilayers comprised of POPC/1% DOPE-NBD/1% DOPE-NTA-Ni (FIG. 14C). Arrows point to specific cells. FIG. 14D-14E are images of PMA activated D10 cells migrating on lipid bilayers comprised of POPC/1% DOPE-NBD/0.5% DOPE-bio alone (FIG. 14D) or bound by SA-(ICAM-1)₂ (FIG. 14E). Figure shows the lipid bilayer of FIG. 14D bound by cells.

FIGS. 15A and 15B are images of lipid bilayers (left) and calcium flux studies (right) of lipid bilayers comprising POPC/1% DOPE-NBD/1% DOPE-bio bound with SA-(ICAM-1)₂ (FIG. 15A) or with SA-(ICAM-1)₂ and SA-(I-Ek-MCC)₂ (FIG. 15B).

FIG. 16 is an image of a POPC/1% DOPE-NBD/1% DOPE-bio lipid bilayer bound by streptavidin-FITC.

FIG. 17A is a schematic of dendritic cell and naive T cell interactions (Kikutani et al. (2003) Nature Reviews 3:159-167) and FIG. 17B is a schematic drawing of interactions involved in APC signaling of T cells (Pardoll (2002) Nature Reviews 2:227-238).

### DETAILED DESCRIPTION OF THE INVENTION

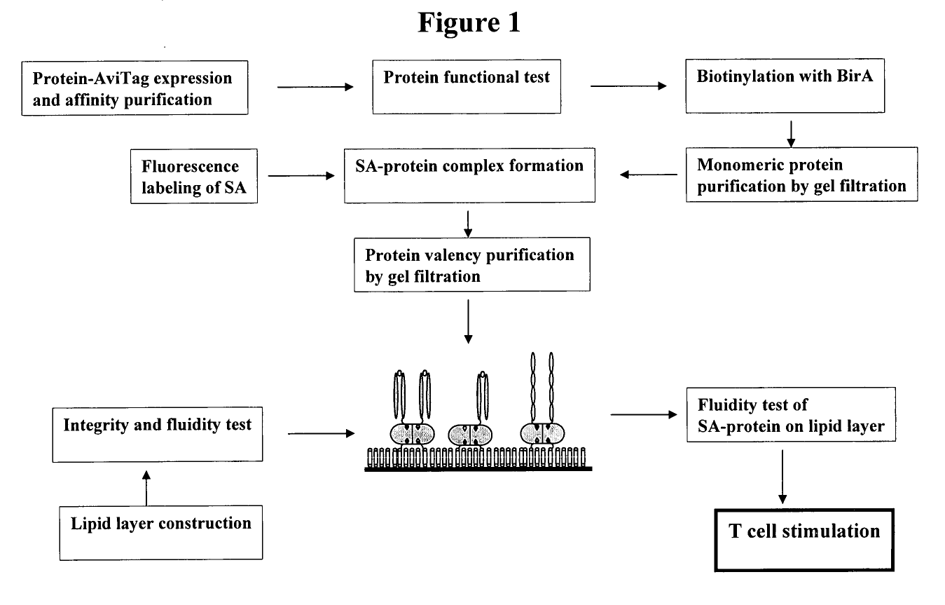

A process of generating a fluid planar lipid layer-based membrane-anchored ligand system with defined ligand valency is summarized in FIG. 1. The process as described herein provides for a system with advantages over traditional approaches including a larger area of membrane homogeneity, mobility of the ligands of interest, guaranteed ligand purity, and an ability to define the ligand valency. Further, methods for preparing a lipid bilayer, which is highly homologous and mobile, by liposome fusion and bilayer spreading are provided. The fluid planar lipid layer-based membrane-anchored ligand system of the instant invention can be used to address, among other things, the ligand valency requirement for receptor activation, ligand distribution at the contact interface, kinetics of ligand-receptor binding, membrane fusion mechanisms, the functional relationships of multiple ligands involved in the same process, and potential modulating ability of a compound on the various ligand-receptor interactions. Potential systems that can be studied with the fluid planar lipid layer-based membrane-anchored ligand system include, but are not limited to: cell adhesion and migration, interactions between T and B cells, interactions between T cells and antigen presenting cells (APCs), and virus-cell interactions.

Additionally, the bilayer systems of the instant application can be employed in the following applications. First, the bilayer system can be employed to determine the valency requirement for interactions between membrane-anchored ligands and receptors. Second, the bilyar system can be used as a platform for a pep-MHC (peptide-MHC) array which can be used to detect T cells bearing specific T-cell receptors (TCRs). Because of the fluidity of pep-MHC in the instant bilayer systems, the instant invention may provide more specific and more accurate detection than pep-MHC fixed on glass or plastic. Third, the bilayer system may be employed as a platform for surface plasmon resonance (SPR) assays. The fluid bilayers of the instant invention may be particularly useful in analysis of binding involving a ligand and more than one membrane receptor. For example, TCR binds to pep-MHC and CD4 or CD8 co-receptors simultaneously. A chip with fluid, membrane-anchored pep-MHC and CD4 or CD8 will provide more accurate and more physiological TCR binding kinetics than analyzing the interaction between pep-MHC and TCR alone. Other examples of ligands and multiple receptors combinations include, without limitation, pep-MHC and costimulatory molecules such as, without limitation, CD28 and CTLA-4; and CD4 and HIV-1 co-receptors such as, without limitation, the chemokines CCR5 and CXCR4. Furthermore, such chips in the SPR assay would be effective for the rapid screening of compounds capable of disrupting the interactions being studied. For example, an SPR chip comprising CD4 and at least one HIV-1 co-receptor would allow for the screening of potential anti-HIV drugs based the compounds ability to disrupt an interaction between the SPR chip and HIV-1 virions or HIV-1 gp120. Fourth, the bilayers of the instant invention may be employed as a potential platform for fast protein separation based on charge differences. Proteins with different net charge may be forced to migrate with different speed or direction when voltage is applied across the membrane. Fifth, the bilayer of the instant invention may be used a platform for 2D crystallography.

The present invention also encompasses kits for use in producing a fluid planar lipid layer-based membrane-anchored ligand system with defined ligand valency to study the valency requirement of a given interation. Such kits comprise lipids; solid supports; and first and second specific binding pair members. The solid support is preferably glass. The first binding pair member is preferably biotin and the second binding pair member id preferably streptavidin.

The kits may further comprise at least one ligand of interest which is optionally functionally linked to a specific binding pair member, appropriate buffers, frozen stocks of host cells, gel filtration materials, and instruction material.

As used herein, an “instructional material” includes a publication, a recording, a diagram, or any other medium of expression which can be used to communicate the usefulness of the composition of the invention for performing a method of the invention. The instructional material of the kit of the invention can, for example, be affixed to a container which contains a kit of the invention to be shipped together with a container which contains the kit. Alternatively, the instructional material can be shipped separately from the container with the intention that the instructional material and kit be used cooperatively by the recipient.

The term “specific binding pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin, biotin/biotin binding agent, Ni2+ or Cu2+/HisTag (6× histidine) and virus-receptor interactions. Various other specific binding pairs are contemplated for use in practicing the methods of this invention, such as will be apparent to those skilled in the art.

The term “antibody” as used herein, includes immunoglobulins, monoclonal or polyclonal antibodies, immunoreactive immunoglobulin fragments, and single chain antibodies. Also contemplated for use in the invention are peptides, oligonucleotides or a combination thereof which specifically recognize determinants with specificity similar to traditionally generated antibodies.

As used herein, “biotin binding agent” encompasses avidin, streptavidin and other avidin analogs such as streptavidin or avidin conjugates, highly purified and fractionated species of avidin or streptavidin, and non or partial amino acid variants, recombinant or chemically synthesized avidin analogs with amino acid or chemical substitutions which still accommodate biotin binding. Preferably, each biotin binding agent molecule binds at least two biotin moieties and more preferably at least four biotin moieties.

As used herein, “biotin” encompasses biotin in addition to biocytin and other biotin analogs such as biotin amido caproate N-hydroxysuccinimide ester, biotin 4-amidobenzoic acid, biotinamide caproyl hydrazide and other biotin derivatives and conjugates. Other derivatives include biotin-dextran, biotin-disulfide-N-hydroxysuccinimide ester, biotin-6 amido quinoline, biotin hydrazide, d-biotin-N hydroxysuccinimide ester, biotin maleimide, d-biotin p-nitrophenyl ester, biotinylated nucleotides and biotinylated amino acids such as Nε-biotinyl-1-lysine.

As used herein, a ligand can be, but is not limited to, receptors, monoclonal or polyclonal antibodies, viruses, chemotherapeutic agents, receptor agonists and antagonists, antibody fragments, lectin, albumin, peptides, proteins, hormones, amino sugars, lipids, fatty acids, nucleic acids and cells prepared or isolated from natural or synthetic sources. In short, any site-specific ligand for any molecular epitope or receptor to be detected through the practice of the invention may be utilized. Preferably, the ligand is a membrane-anchored protein. The ligand may also be a derivative of a membrane-anchored protein, such as a soluble extracellular domain. A ligand can be a receptor involved in receptor-receptor cellular interactions such as TCR binding to the MHC receptor.

The ligands of the instant invention can be expressed and purified by any method known in the art. In a certain embodiment, the proteins are expressed by a baculovirus-based insect expression system or a mammalian expression system. Fifteen residues of AviTag™ peptide may be added to the C-terminals of all of the molecules. The lysine residue in the AviTag™ (Avidity, CO) can be specifically biotinylated by BirA enzyme (Avidity, CO). The proteins may also be designed to be secreted into the supernatant of the cell culture.

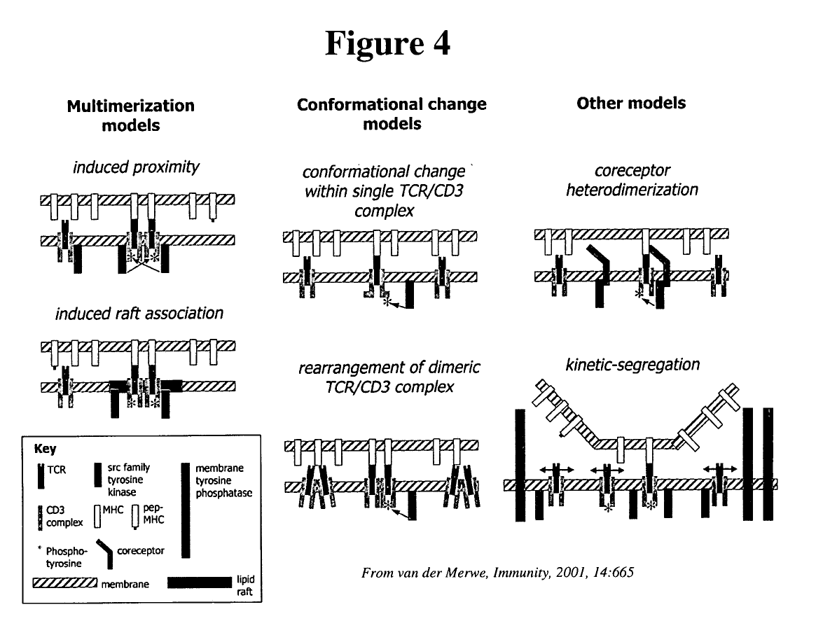

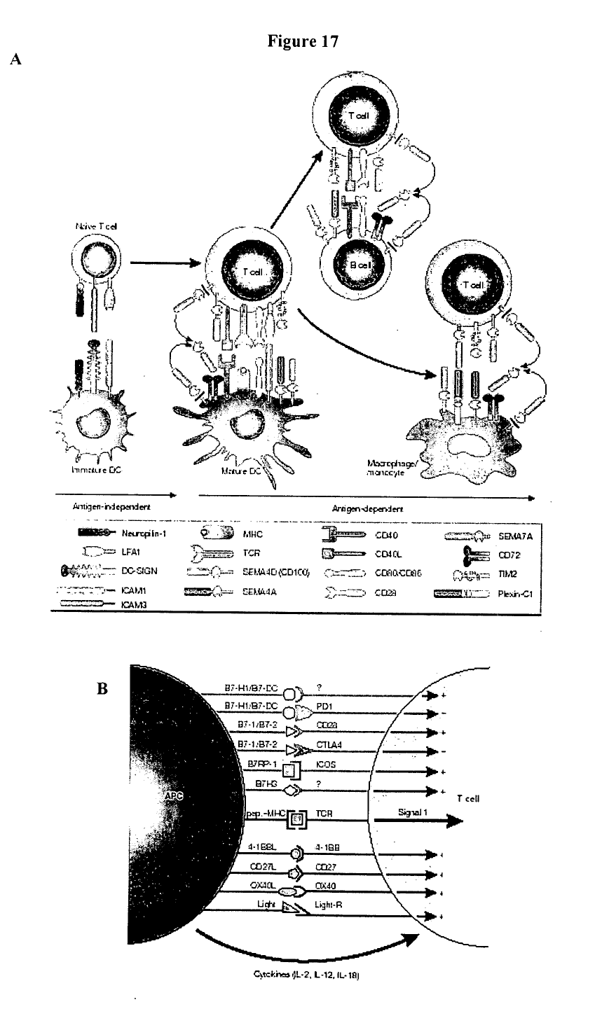

The ligands, as noted hereinabove, can be any protein or peptide. Preferably, the proteins are cell membrane proteins involved in ligand-receptor interactions. Such interactions between membrane anchored ligands and receptors commonly occur in the immune system. For example, the critical event of T lymphocyte (T cell) activation is a result of membrane-membrane contact between T cells and antigen presenting cells (FIG. 4). A variety of ligand-receptor interactions take place between the two opposing membranes, including, major histocompatibility complex (MHC)-peptide and T cell receptor (TCR), LFA-1 and ICAM-1, CD2 and CD48, as well as B7 or CTLA-4 and CD28. Examples of other ligands are provided in FIGS. 17A and 17 B. Understanding the valency requirements of these interactions will facilitate the design of therapeutics that enhance or inhibit the immune response to certain antigens. The instant invention can also be used as a tool to study the subtle differences in T cell intracellular signaling pathways induced by agonist and antagonist antigens. The artificial membrane system provides a clean physiological setting to test the subtle differences without using native antigen presenting cells that often complicate biochemical analyses. Identification of these differences may also provide new targets of therapeutic intervention.

While streptavidin-biotin interactions are exemplified throughout the specification and examples, specific binding pair members as described hereinabove may be employed in place of streptavidin and biotin in the methods of the instant invention. Furthermore, more than one set of specific binding pairs can be employed, particularly when more than one ligand is attached to the membrane surface.

Traditional pep-MHC-streptavidin tetramer technology can also be used to screen T cells of certain pep-MHC specificity. However, T cells with the same specificity may or may not be activated by the same antigen stimulation. To study immune responses (e.g. responses to vaccination [viral or cancer vaccines], immune tolerance, autoimmunity), it is important to discriminate T cells based on their responsiveness to antigen. Using calcium flux by microscopy as an indicator for T cell activation, the instant invention also provides a screening assay to quantify primary T cells responsive to a specific antigen. An alternative approach comprises the use of biotinylated pep-MHC and co-stimulatory molecules coupled onto a streptavidin coated chip. Because of the lack of pep-MHC fluidity, such chips may not be as accurate as the lipid bilayer system disclosed herein.

In addition to the ligand-receptor interactions of the immune system, interactions between viral proteins and their cellular receptors can be studied. Of particular interest is the interaction of the human immunodeficiency virus (HIV) envelope glycoproteins with its cellular receptors. The instant invention can be used to study the mechanism of virus entry thereby identifying therapeutic agents that may block this process.

Other examples of ligand-receptor interactions of interest which are amenable to analysis using the membrane system of the invention, but are not limited to, 1) Interactions employed during cell migration, which include cell-cell contacts and cell-extracellular matrix contacts involved in T cells, dendritic cells, neurons, fibroblasts and other cell types migration. 2) Interactions involved in the synapse-like structure formed at the contact interfaces of T cells and antigen presenting cells and the interface of contacting neurons may also be studied. The synapses feature large scale re-distribution of membrane-anchored molecules that are critical for information transfer. The instant invention allows the formation and function of synapses to be readily observed and studied with real time microscopy.

After purification and biotinylation, the proteins of interest can be further purified by gel filtration with a suitable medium to eliminate spontaneously formed dimers or oligomers. Protein-streptavidin complexes are prepared by mixing the biotinylated proteins with streptavidin (or other biotin binding agents), which can be pre-labeled with fluorescence tags or other detectable agent. Protein-streptavidin complexes of different valencies (one to three proteins per streptavidin) are made by adjusting the molar ratio of streptavidin to protein accordingly when mixing the streptavidin with the biotinylated protein. Size purification using gel filtration chromatography after binding of the biotinylated protein to the streptavidin can be employed to purify only the complexes with the desired valency. Importantly, at least one biotin binding site on the streptavidin molecule should be left vacant in order to anchor the complex to the biotinylated lipid. If desired, hybrid complexes of one streptavidin with two or three different ligands can also be prepared by sequentially forming SA-ligand-1 monomers, size purifying, adding ligand-2, size purifying, adding ligand-3, and size purifying. Valency of the SA-pep-MHC can be guaranteed by gel filtration, Western blot, and if necessary, Biacore or SPR binding assays.

According to one aspect of the instant invention, lipid bilayers can be prepared by liposome fusion and bilayer spreading as exemplified in Example III. Briefly, small unilamellar vesicles (SUV) are produced by the evaporation of a solvent containing the desired lipids. The lipids are subsequently hydrated, vortexed, and sonicated. Contaminants of the SUV are removed by centrifugation. Lipid bilayer formation occurs by fusing the SUV to a clean support, preferably glass, at 4° C. and flushing under water with a stream of water. Part of the lipid bilayer is then exposed to the air to destroy the exposed bilayer. The remaining lipid bilayer is then transferred to a 25° C. environment to allow for warming and spreading of the bilayer. It is this newly formed lipid bilayer portion that is highly fluid and homogeneous and therefore a preferred lipid bilayer system for the methods of the instant invention.

A similar method for the preparation of lipid bilayers by liposome fusion and bilayer spreading was disclosed by Cremer et al. (J. Phys. Chem. B (1999) 103:2554-2559). The method described hereinbelow, however, provides for a gradual increase in temperature during the lipid bilayer spreading step. Cremer et al. teach destroying part of the lipid bilayer at room temperature and instantly allowing for the spread of lipid. The gradual increase in temperature in the instant invention is believed to provide a more fluid and homogeneous lipid bilayer than the method of Cremer et al.

According to another aspect of the instant invention, supported planar lipid layers can also be prepared by three different approaches based on the Langmuir-Blodgett technique. In the following descriptions, 1-Palmitoyl-2-Oleoyl-sn-Glycero-3-Phosphocholine (POPC) is employed for its excellent fluidity at 25-37° C. and its uncharged polar head at physiological pH, but other lipids can be employed. The SA-protein complexes are tethered to a small fraction of 1,2-Dioleoyl-sn-Glycero-3-Phosphoethanolamine-N-(Cap Biotinyl) (DOPE-biotin) doped into the POPC-based layer though other biotinylated lipid can be employed. Fluorescent 1,2-Dioleoyl-sn-Glycero-3-Phosphoethanolamine-N-(Lissamine Rhodamine B Sulfonyl) (DOPE-Rhodamine) or other fluorescently tagged lipids can be used to check the integrity, homogeneity, and fluidity of the lipid layers because of its structural similarity with DOPE-biotin. All lipids can be purchased from Avanti Polar Lipids (Alabaster, Ala.). General lipid bilayer techniques are previously described (Tamm and McConnell (1985) Biophys J. 47(1):105-113; Kalb, E. et al. (1992) Biochim Biophys Acta. 1103(2):307-316; von Tschamer and McConnell (1981) Biophys J. 36(2):421-427; Subramaniam, S. et al. (1986) Proc Natl Acad Sci USA 83(5):1169-1173).

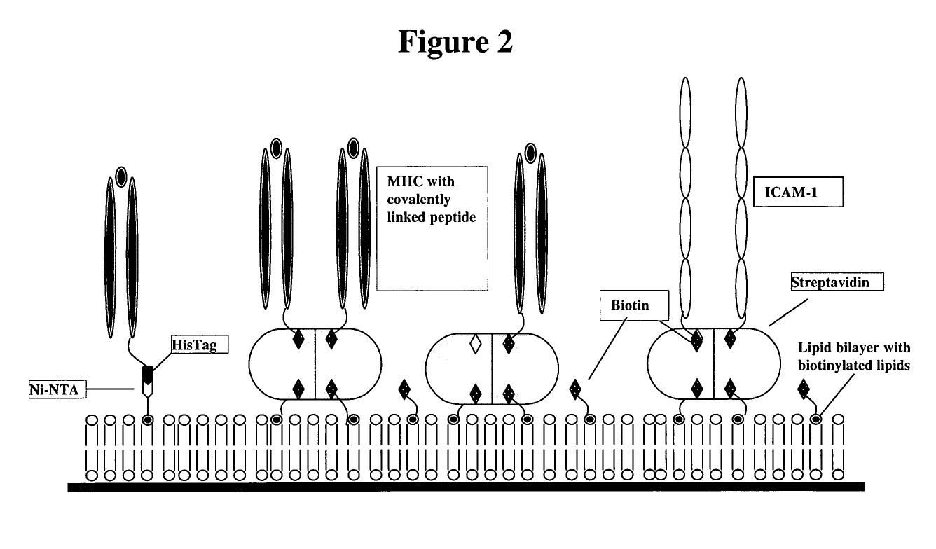

In the first approach, the bilayer approach (FIG. 2), both layers are deposited on a glass coverglass using the Langmuir-Blodgett technique. First, lipid monolayers are formed by spreading 1 mg/ml solutions of lipids in chloroform/hexane (1:1) at near-zero surface pressure at the air-water interface of a Langmuir-Blodgett trough (μTrough S, Kibron, Helsinki, Finland). The subphase is 10 mM Tris-acetic acid (pH 5.0) made from deionized water. The solvent is allowed to evaporate for 30 min before the monolayers are compressed. Monolayers are then transferred to glass coverslips at a pressure of 30 mN/m. This is accomplished by first forming a monolayer at ˜0 pressure and compressing it to 30 mN/m. The cleaned coverslip is then quickly (20 mm/min) immersed through the monolayer vertically and into the subphase. No lipid should be transferred at this step, and the surface pressure should be virtually unchanged. The coverslip is then withdrawn from the subphase at a rate of 2.5 mm/min while a surface pressure of 30 mN/m is maintained with an electronic feedback circuit. A single monolayer is formed in this step on the surface. The second monolayer is transferred by horizontally pushing the coverglass, with the first layer facing down, through the film on top of 10 mM Tris-HCl, 150 mM NaCI, pH 7.5 buffer at 30 mN/m.

In the second approach, the monolayer approach, the first layer is prepared by derivatizing the coverslip with 0.1% decyltrichorosilane (DTS, C10) (United Chemical Technologies, PA). In this way a hydrophobic ten carbon acyl chain is covalently attached to the glass surface, thus providing a stable support for the second layer, which can then be deposited by the Langmuir-Blodgett technique as described above. The top layer of such a system has been shown to be fluid and antibodies bound to the hapten linked to the lipid head group can diffuse freely laterally.

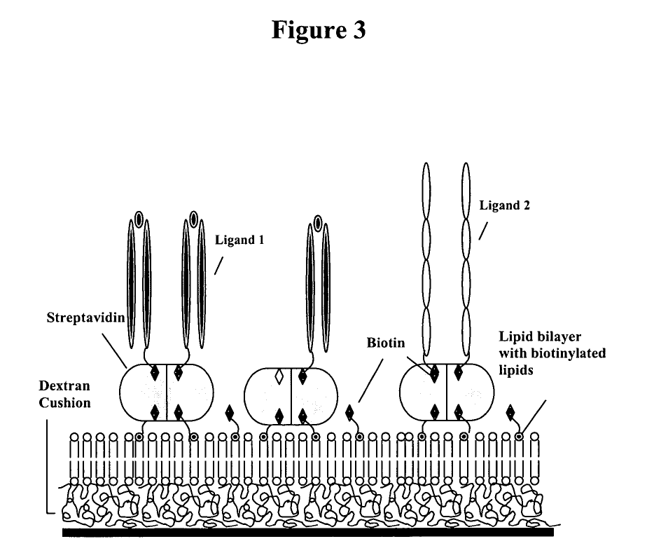

The third approach is to prepare a Dextran-cushioned lipid bilayer (FIG. 3). It is similar to the first approach except that the lipid bilayer will be deposited on a Dextran-cushioned coverglass. To attach the Dextran cushion, the coverglass is first be silanated with epoxysilane ((3-(2,3-epoxypropoxy)propyl)trimethoxysilane, Sigma). The coverslips are then incubated in 30% Dextran T500 (Amersham Biosciences) solution overnight to couple Dextran polymers to the coverglass through reactions between Dextran's hydroxyl groups and the epoxy groups. The coverslips are then be washed in DI water for 48 hrs with multiple water changes to eliminate non-specifically bound Dextran polymers.

The coverglass with the lipid bilayer is transferred under water into a culture dish with PBS buffer. After blocking with 1% BSA for 30 min, the streptavidin-protein complexes are added to the buffer at different concentrations for 30 minutes at room temperature. Such BSA blocking is not required for the Dextran-cushioned lipid bilayer approach. Notably, the streptavidin-protein complexes can all consist of the same protein or complexes consisting of different proteins may be added to the lipid layer. The surface density of bound complexes can be directly determined by atomic force microscopy, ELISA, or radioimmunoassay. Additionally, the fluidity of the membrane can be assayed by fluorescence recovery after photobleaching (FRAP). This assay can quantitatively assess both diffusion rate and mobile fraction (Smith, L. M. et al. (1980) Biochemistry. 19(25): 5907-5911). The same can be done for the lateral mobility of tethered fluorescence labeled SA-protein. In addition, the mobility of tethered SA-protein complexes can be tested by observing the Brownian motion of small latex beads (0.1 mm in diameter) coated with monoclonal antibodies specific for the protein (Lee, G. M. et al. (1991) Proc Natl Acad Sci USA. 88(14): 6274-6278).

Lipid bilayers of the instant invention may comprise a lipid content that most closely mimics the cellular system being studied. Further, integral membrane proteins may be inserted into the lipid bilayers to more closely mimic the cellular surface.

Additionally, fluorescence resonance energy transfer (FRET) can be used to determine the distance between ligand and receptor molecules, ligand molecules, and receptor molecules. The resolution for traditional fluorescence microscopy is 200 nm, far lower than the sensitivity required to look for molecular interactions. FRET provides a means of increasing the resolution of light microscopy to 10⁻¹ nm (Gordon, G. W. et al. (1998) Biophys J. 74(5): 2702-2713). The principle of resonance energy transfer is based on the ability of a higher energy donor fluorophore to transfer energy directly to a lower energy acceptor molecule, causing sensitized fluorescence of the acceptor molecule and simultaneous quenching of the donor fluorescence. The efficiency of energy transfer (E) is highly dependent on the distance (r) between the donor and acceptor fluorophores, as described by the Förster equation: E=R₀⁶/(R₀⁶+r⁶), where r is the distance between the fluorophores and the Förster radius (R₀) is the distance at which the efficiency of energy transfer is 50% of maximum. Because the E is inversely correlated with the r⁶, usually only when the distance between donor and acceptor fluorophores is within 10 nm can the energy transfer be observed. FRET can be observed as decrease of donor emission, acceptor emission after donor excitation, or increased donor emission after photobleaching the acceptor (Kenworthy and Edidin (1998) J Cell Biol. 142(1): 69-84). FRET can be conducted on cells and the lipid bilayer of the instant invention. Specific antibodies and Cy3 (donor fluorophore) or Cy5 (acceptor fluorophore) labeled secondary antibodies can be used for fixed cells. Additionally, streptavidin can be labeled with Cy3 or Cy5 so that the distance between them can be measured.

The following examples illustrate various aspects of the present invention. They are not intended to limit the invention in any way.

### EXAMPLE I

### Lipid Monolayer

In collaboration with Dr. John Kappler at the National Jewish Medical and Research Center, the extracellular domains of murine I-Ek-MCC (Crawford, F. et al. (1998) Immunity. 8(6):675-682; Kozono, H. et al. (1994) Nature. 369(6476):151-154), I-Ak-CA (Khandekar, S. S. et al. (1997) Mol Immunol. 34(6): 493-503), ICAM-1 (Cobb, R. R. et al. (1992) Biochem Biophys Res Commun. 185(3): 1022-1033), B7.1 (Nagarajan, S. and P. Selvaraj (1999) Protein Expr Purif. 17(2): 273-281) and CD48 were expressed by a baculovirus-based insect expression system (Invitrogen, CA). With the exception of CD48, all of these proteins have previously been successfully expressed in baculovirus-based insect cells and proven functional. I-Ek-MCC and I-Ak-CA are MHC class II molecules covalently linked with the moth cytochrome c peptide (residues 88-103) and conalbumin peptide (residues 134-146), respectively. Fifteen residues of AviTag peptide were added to the C-terminals of all of the molecules. The lysine residue in the AviTag can be specifically biotinylated by BirA enzyme (Avidity, CO; Crawford, F. et al. (1998) Immunity. 8(6):675-682). I-Ek and I-Ak with null peptides (K99A and M7M, respectively) were also generated as negative controls (Reay, P.A. et al.(1994) J Immunol. 152(8): 3946-3957; Dittel, B. N. et al. (1997) J Immunol. 158(9): 4065-4073). To facilitate pairing of I-Akα and β chain, the acidic and basic half of leucine zipper protein will be between the I-Ak and the AviTag (Khandekar, S. S. et al. (1997) Mol Immunol. 34(6): 493-503).

The expression of ICAM-1, B7.1, and I-Ak-CA were confirmed by Western blot (data not shown). Briefly, 20 μl supernatant of Hi5 insect cell culture, day 0 to day 4 post infection by baculoviruses encoding ICAM-1, was blotted with ICAM-1 specific YN1.7.4 antibody. ICAM-1 positive CH27 cell lysate were used as a positive control. The bands higher than 97KDa are dimer and oligomers of ˜60KDa ICAM-1. The lower MW of expressed ICAM-1 compared to native ICAM-1 (˜95KDa) is probably due to incomplete glycosylation. Supernatants of Hi5 cell culture, day 0 to day 3 post infection by baculoviruses encoding I-Ak-CA, were blotted with I-Ak β chain specific 10.2.16 antibody. Supernatants of Hi5 cell culture, day 0 to day 2 post infection by baculoviruses encoding B7.1, were blotted with B7.1 specific 16-10A1 antibody.

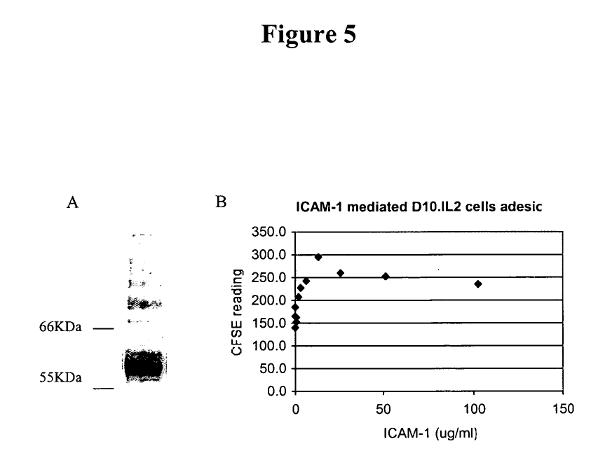

ICAM-1-AviTag-HisTag was purified with a Ni²⁺-NTA column (FIG. 5A) and its function tested with an adhesion assay (FIG. 5B). Briefly, the purification of ICAM-1-AviTag-HisTag was demonstrated by Coomassie Blue staining of ICAM-1 purified on a Ni2+ column. The higher MW bands are likely oligomers of ICAM-1. The function of the ICAM-1 was tested by dose response of adhesion of D10.IL2 cells to 96 well ELISA plates coated with purified ICAM-1. Plates were coated with different concentrations of ICAM-1 by 2× serial dilution in TBS buffer (pH 8.0) at 4° C. overnight. The plate was blocked with 3% BSA in TBS and washed with PBS. 10⁵ CFSE (carboxyfluorescein succinimidyl ester) labeled D10.IL2 T cells were added to each well and incubated at 37° C. incubator for 45 min. The plate was then inverted and cultured for another 45 min so that the cells that did not adhere drop from the plate bottom by gravity. The media and non-adherent cells were removed by paper towel adsorption and the cells remaining were lysed with 2% Tween-20 in PBS. The plate was read using a fluorescence plate reader with excitation at 485 nm and emission at 527 nm.

I-Ak-CA was purified with the leucine zipper specific monoclonal antibody 2H11 by immunoaffinity chromatography (data not shown). The purification was verified by coomassie blue staining of purified I-Ak CA on polyacrylamide gel electrophoresis (PAGE). Fractions were collected during elution of anti-leucine zipper antibody (2H11) conjugated affinity column. Higher and lower bands were identified as the I-Ak β and α chains, respectively. The purified protein was confirmed to be a heterodimer of α and β by gel filtration (data not shown). The calculated MW of I-Ak-CA is about 56KDa. The purified I-Ak-CA was tested to be functional when used to stimulate D10.IL2 cells (data not shown). Specifically, the functional test of purified I-Ak-CA comprised coating 96 well tissue culture plates with I-Ak-CA or BSA at ˜30 μg/ml at 4° C. overnight. The plates were washed 3 times with PBS and 2.5×10⁵ D10.IL2 cells were added with RPMI-10% FBS containing 50 U/ml IL-2 and 20 μg/ml brefeldin A. Cells were incubated at 37° C. for 8 hrs, fixed with 2.5% formaldehyde, permeabilized with PBS containing 1% BSA and 0.1% saponin, and stained for IL-4 with APC conjugated antibody 11H11. I-Ak-CA, but not BSA, increased IL-4 expression.

The monolayer approach, coating a layer of POPC on top of derivatized coverglass, was employed in this example. Judging by DOPE-Rho doped in the POPC, the monolayer is homogeneous and stable for at least 4 days under PBS. The POPC monolayer doped with 2 mol % DOPE-Rho as a marker was readily visible by fluorescent microscopy. POPC monolayers containing 10 mol % DOPE-bio or DOPC were blocked with PBS-1% BSA for 30 min at room temperature. After adding 10 ug/ml gel filtration purified SA-(ICAM-1-bio)₂ in PBS-1% BSA for 1 hr at room temperature, the monolayer was washed with DMEM-10% FBS 10 times. The monolayer was then stained with ICAM-1 specific antibody YN/1.7.4 and secondary goat anti-rat-FITC. Notably, the monolayer with DOPE-bio showed streptavidin binding capacity.

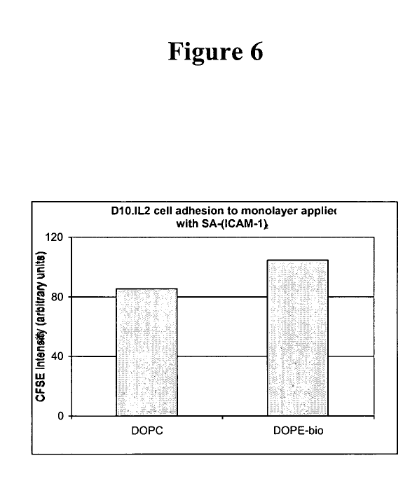

ICAM-1 molecules that were biotinylated by BirA enzyme formed SA-(ICAM-1)ₙ complexes (wherein n is the number of ICAM-1 molecules per streptavidin molecule) when mixed with streptavidin. Change of the molar ratio of ICAM-1-bio to SA resulted in complexes of different molecular weights as judged by chromatographic behavior in gel filtration. The purified dimeric SA-(ICAM-1)₂ binds to monolayers with DOPE-bio, but not to monolayers without DOPE-bio, as detected by anti-ICAM-1 antibodies. D10.IL2 cells showed increased adhesion to monolayers with bound SA-(ICAM-1)₂ (FIG. 6). Briefly, after SA-(ICAM-1-bio)₂ was applied to POPC monolayers containing DOPE-bio or DOPC, fluorescently labeled D10.IL2 cells were added in DMEM-10% FBS medium for 30 min. The coverslip with attached cells was then inverted and incubated for one hour. The cells were lysed and analyzed as described hereinabove. The limited increase of D10 adhesion is probably largely due to a high background adhesion of D10 to BSA blocked monolayer without SA-(ICAM-1)₂. Additionally, the LFA-1 molecules on D10.IL2 cells were not significantly in an activated state.

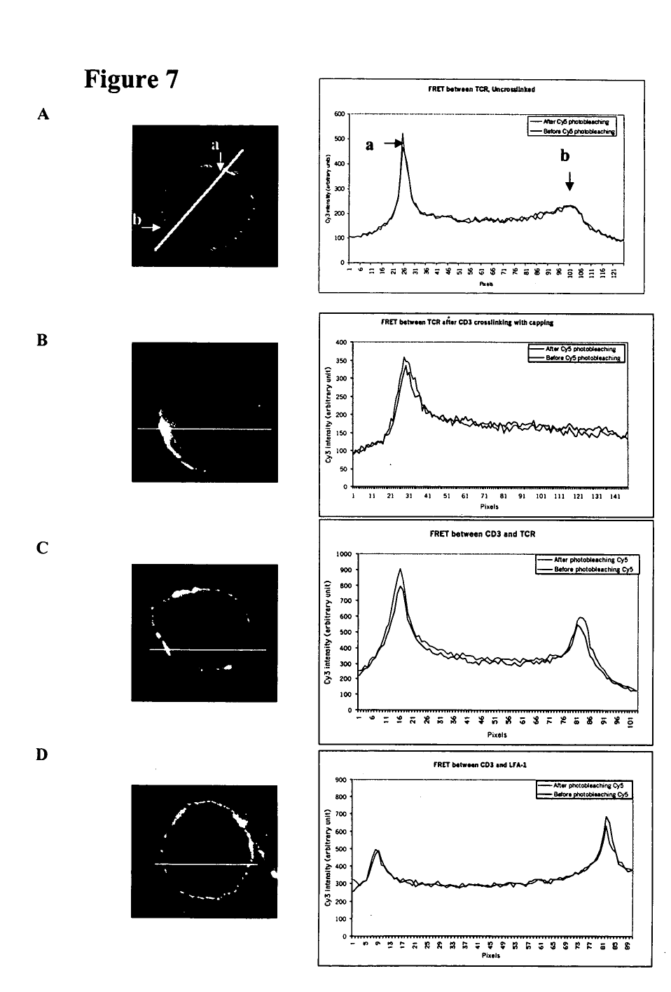

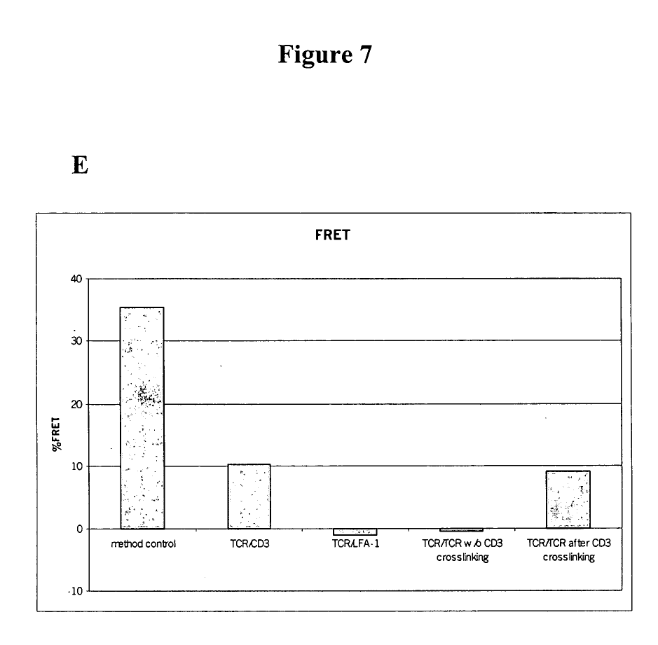

Using acceptor photobleaching recovery FRET, we have tested the presence of TCR dimers on D10.IL2 T cell clones before and after CD3 is crosslinked with anti-CD3ε antibodies (FIG. 7). TCR proximity was analyzed by FRET. Specifically, resting D10.IL2 cells were fixed with 2.5% formaldehyde and incubated with a mixture of KJ16 (rat) and F23.1 (mouse). Both of these antibodies recognize TCR Vβ 8.2 and block each other for binding, as determined by FACS. A mixture of Cy3 labeled donkey anti-rat (minimum crossreaction to mouse) and biotin labeled donkey anti-mouse (minimum reaction to rat) was used as secondary antibodies. SA-Cy5 was added last. Controls using secondary antibodies and SA-Cy5 showed no non-specific staining or crossreaction between antibodies. An image of Cy3 was first captured before Cy5 was photobleached until 95% of intensity was lost. A group of Cy3 images were then taken along the z-axis to ensure perfect matching to the pre-photobleaching images. Cy3 images of pre-photobleaching (shown as green) and post-photobleaching (shown as red) were merged and aligned. A line was drawn to mark a narrow band of pixels across the T cell. The intensity of the marked pixels calculated by the Slidebook software was plotted. TCR/CD3 of D10.IL2 cells were also crosslinked with anti-CD3ε antibody (2C11) and goat anti-hamster antibody (FIG. 7B). Resting T cells (FIG. 7C) were also stained with a mixture of anti-TCR Vβ 8.2 antibody F23.1 and anti-CD3ε antibody 500A2 (hamster), then stained with corresponding non-crossreacting secondary antibodies labeled with Cy3 (for CD3ε) or Cy5 (for TCR). Additionally, resting T cells were stained with a mixture of anti-CD3ε antibody 500A2 and anti-LFA-1 antibody I21/7 (rat), then stained with corresponding non-crossreacting secondary antibodies labeled with Cy3 (for LFA-1) or Cy5 (for CD3ε) (FIG. 7D). The Cy3 mean intensity before and after Cy5 photobleaching was calculated based on all pixels shown in FIGS. 7A-7D (FIG. 7E). % FRET was calculated as (Cy3 intensity after Cy5 photobleaching—Cy3 intensity before Cy5 photobleaching)/(Cy3 intensity before Cy5 photobleaching). The negative value is very likely caused by photobleaching of Cy3 during image capturing after Cy5 photobleaching. Data are representative of 8 cells for each group.

Note from FIG. 7A to 7D, FRET was shown as the difference between the red and blue lines indicating the intensity of Cy3 along the white line in the images after and before Cy5 photobleaching, respectively. In FIG. 7E, the % FRET was derived from all the pixels shown in the image. When the whole TCR population was evaluated, no FRET was detectable on resting D10.IL2 cells (before TCR crosslinking) (FIG. 7A, 7E). However, after TCR crosslinking and resultant TCR capping, about 10% of FRET was observed (FIG. 7B, 7E). Although the % FRET is not very high, it is comparable to our positive control (FIG. 7C, 7E), FRET between TCRβ and CD3ε, and >2% is considered significant in this type of assay (Szaba, G., Jr. et al. (1992) Biophys J. 61(3): 661-670). However, when small areas were examined individually, FRET between TCRs can be detected at areas where TCRs are highly concentrated (FIG. 7A, arrow a). FRET between CD3ε and LFA-1 was used as a negative control. These results are at odds with a previous study measuring FRET between FITC and PE with flow cytometry, which reported 15%-20% FRET between TCRs without TCR or CD3 crosslinking. One major difference is that the cells were fixed before staining, while live cells were stained with antibody at 4° C. and then measured in the previous study. It is possible that during live cell staining, even at 4° C., TCR can be clustered by antibodies. Also, the “bleeding through” between FITC and PE channels in flow cytometer may also complicate data acquisition and interpretation. The present result indicates that close proximity between TCRs is not a universal phenomenon on the T cell surface. Rather, monomeric TCRs are distributed in different densities across the T cell surface. We are not certain as to the significance of FRET detected in TCR highly enriched areas. Because we used TCR specific antibodies and labeled secondary antibodies, depending on the orientation of these interactions, the antibodies may have increased the theoretical R₀ of FRET between Cy3 and Cy5 to certain degree. Taken together, our results do not support the presence of pre-formed TCR dimers on the T cell surface.

Fluidity assays of the POPC monolayer were also performed. Incorporated DOPE-FITC lipids (2%) were determined to be fluid throughout the lipid layer (data not shown). Streptavidin-FITC attached to POPC lipid layers with 10% DOPE-bio was shown to have low levels of fluidity (data not shown).

### EXAMPLE II

### Lipid Bilayer

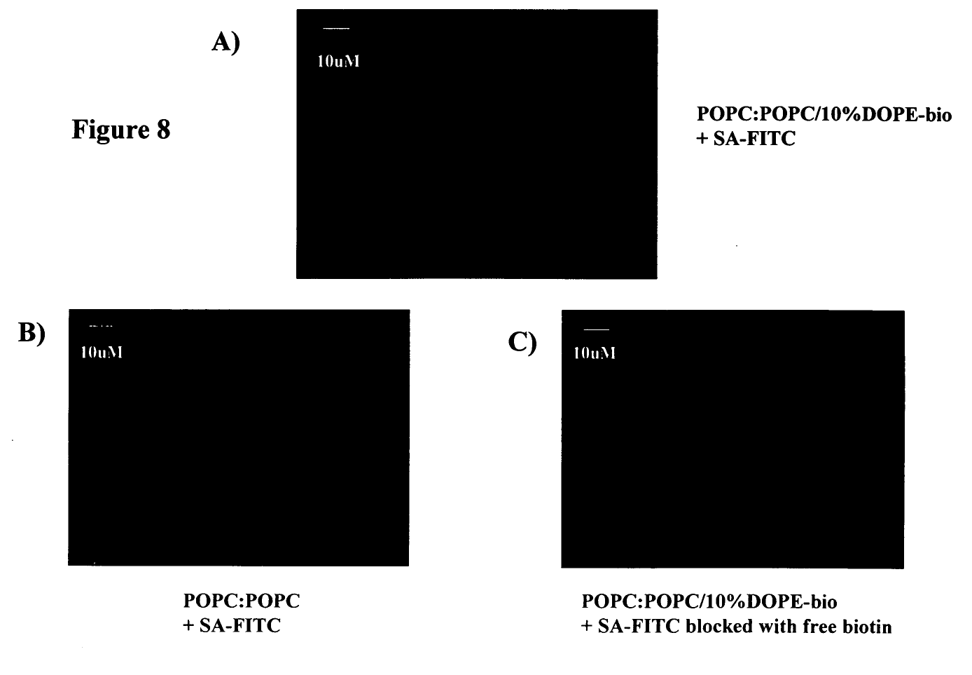

Dextran-cushioned lipid bilayers were prepared as described above. Judging by DOPE-Rho doped in the POPC, the monolayer is homogeneous and stable (data not shown). Additionally, Dextran-cushioned lipid bilayers containing DOPE-bio were determined to bind FITC conjugated straptavidin specifically without BSA blocking (FIG. 8A-8C).

Fluidity assays of the Dextran-cushioned lipid bilayer were also performed. Incorporated DOPE-FITC lipids (2%) were determined to be fluid throughout a lipid bilayer (POPC:POPC/2 mol % DOPE-FITC; data not shown). Streptavidin-FITC attached to a POPC:POPC/10% DOPE-bio bilayer was also shown to fluid within the lipid bilayer (data not shown).

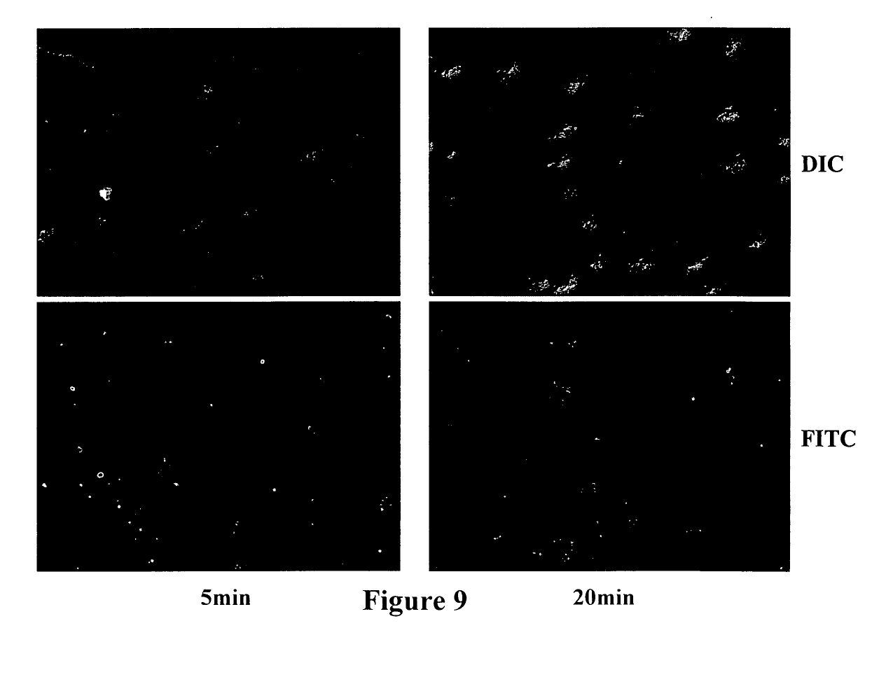

Additionally, T-cells were capable of being recruited to the lipid bilayer through streptavidin FITC with biotinylated antibodies to CD25 (FIG. 9). Notably, T cells, as a control, were unable to adhere to POPC bilayers in the presence of 10% FBS (fetal bovine serum). Therefore, the generated Dextran-cushioned POPC bilayer provides a fluid and inert surface.

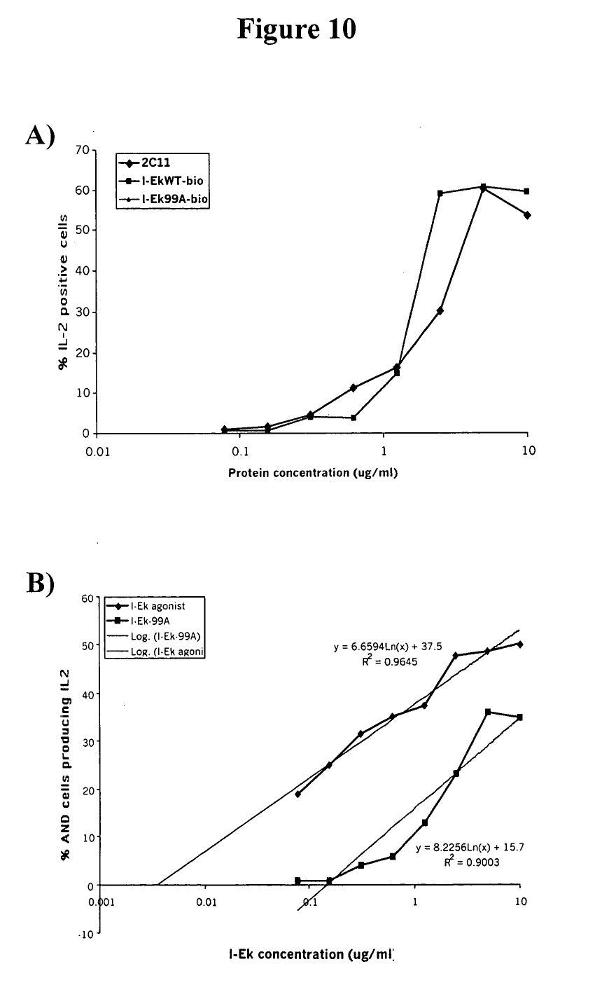

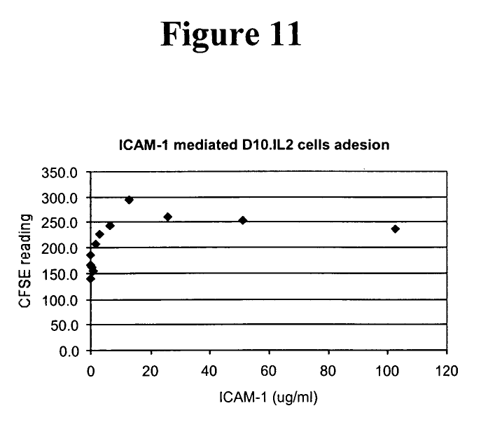

Purification of I-Ak-CA and I-Ek-MCC was confirmed by Coomassie blue staining and gel purification (data not shown). FIGS. 10A and 10B represent the results from functional assays of I-Ek-MCC with wild-type and null peptides. The production and purification of ICAM-1-AviTag-HisTag was confirmed by Coomassie blue staining and the functionality of ICAM-1-AviTag-HisTag was shown by ability to adhere D10 cells (FIG. 11). Purification of B7.1-AviTag and CD48-AviTag-HisTag was confirmed by Western blot (data not shown).

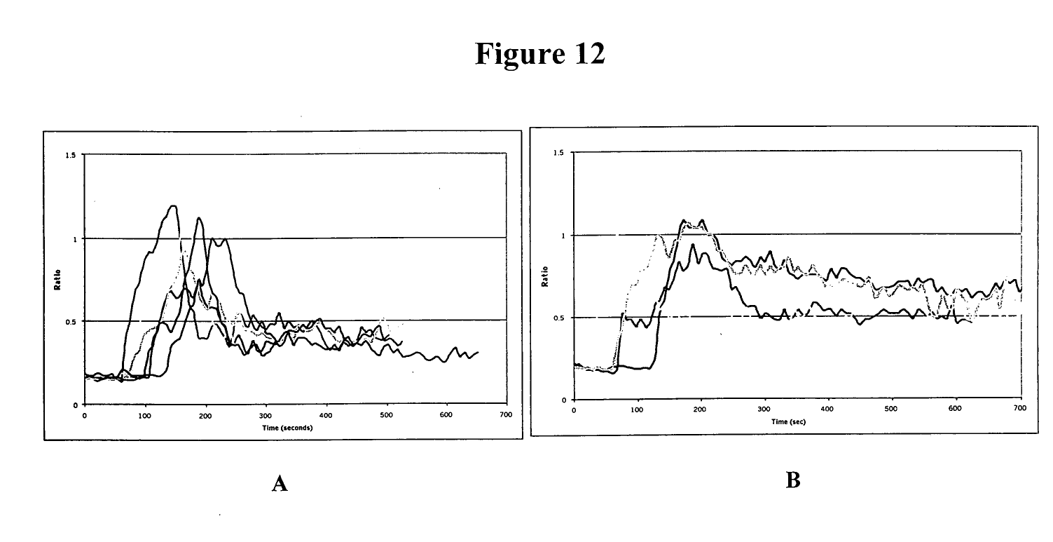

Importantly, streptavidin-I-Ek multimers anchored on a POPC/POPC/DOPE-bio bilayer in media containing 10% FBS were able to induce calcium flux in T cells (FIG. 12A). FIG. 12B depicts the calcium flux induced by CH27 cells pulsed with PCC in medium containing 10% FBS. Streptavidin-ICAM-1 multimers anchored on a POPC bilayer induce T cell adhesion and migration, but not calcium flux (data not shown).

### EXAMPLE III

### Lipid Bilayers by Liposome Fusion and Bilayer Spreading

Lipid bilayers prepared by liposome fusion and bilayer spreading were created by the following method.

Glass coverslips (22 mm diameter circular glass coverslips from Fisher Scientific, Hampton, N.H.) were washed in 10% hot Contrad® 70 (Decon Labs; Bryn Mawr, Pa.) in a bath sonicator for 30 minutes and then rinsed exhaustively with deionized (DI) water. The glass coverslips were dried at 150° C. and subsequently soaked in chromic sulfuric acid solution (Fisher Scientific) overnight followed by rinsing under flow of DI water overnight. The coverslips are finally dried at 150° C.

Small unilamellar vesicles (SUV) were prepared by the following methods. Two mg of total lipids of 1-Palmitoyl-2-Oleoyl-sn-Glycero-3-Phosphocholine (POPC), 1% 1,2-Dioleoyl-sn-Glycero-3-Phosphoethanolamine-N-(7-nitro-2-1,3-benzoxadiazol-4-yl) (Ammonium Salt) (DOPE-NBD) and desired percentage of 1,2-Dioleoyl-sn-Glycero-3-Phosphoethanolamine-N-(Cap Biotinyl) (Sodium Salt) (DOPE-biotin) or 1,2-Dioleoyl-sn-Glycero-3-{[N(5-Amino-1-Carboxypentyl)iminodiAcetic Acid] Succinyl} (Nickel Salt) (DOGS-NTA-Ni) were mixed in a glass test tube. All lipids may be obtained from Avanti Polar Lipids and used without further purification. The chloroform solvent was evaporated under flow of nitrogen and the lipids were further dried for 2 hrs under a vacuum. The lipids were then re-hydrated in 3 ml 150 mM NaCl, 5 mM HEPES buffer pH 7.4 by four freeze-thaw cycles and intensive vortexing. The lipid suspension was sonicated with a probe sonicator (VibraCell, Sonics & Materials, Inc.; Newtown, Conn.) at 30% output for 30 minutes with 10 seconds on and 10 seconds off pulsing while submerged in ice water. The resulted lipid solution was first centrifuged at 12,000 g to eliminate titanium particles from the sonicator probe. The solution was then spun at 40,000 rpm for 2 hours with an ultracentrifuge (Beckman Coulter; Fullerton, Calif.) to separate the small unilamellar vesicles (SUV) from the remaining large multilamellar vesicles. The SUV are stored at 4° C. until use.

The lipid bilayer was formed as follows. 600 μl of 4° C. SUV were diluted to 100 μg/ml and added to a pre-cooled coverglass and allowed to fuse for 15 minutes at 4° C. The coverglass was then submerged in 2L, 4° C., DI water and flushed under water with a stream of 4° C., DI water at about 0.5L/min/cm². Half of the coverglass was then briefly exposed to air to destroy the bilayer. The coverglass was then transferred to a dish containing approximately 3 ml, 4° C., Dulbecco's PBS (without Ca²⁺, Mg²⁺ pH 7.4; DPBS). The dish was transferred to 25° C. and allowed to sit for 30 minutes to 1 hour. The bilayer spreads as the DPBS temperature gradually increases.

Ligands were bound to the bilayer by first blocking with 10 mg/ml BSA in HSPG (150 mM NaCl, 5 mM KCl, 2 mg/ml glucose, 10 mM HEPES pH 7.4). Ligand-streptavidin complexes or ligands with 6×His tag in the blocking buffer are added and incubated for 30 minutes before extensive washing.

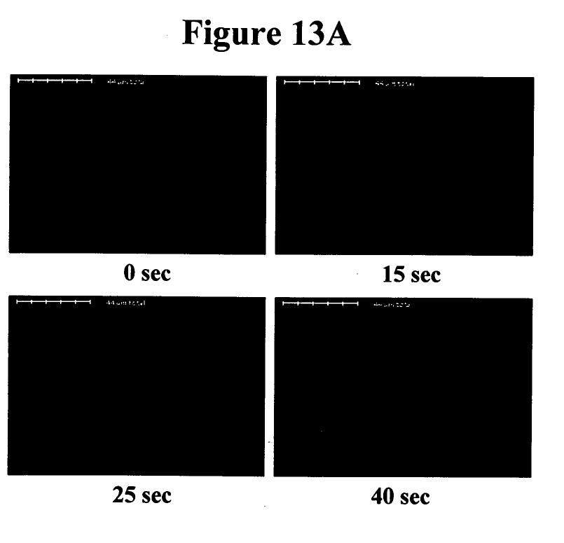

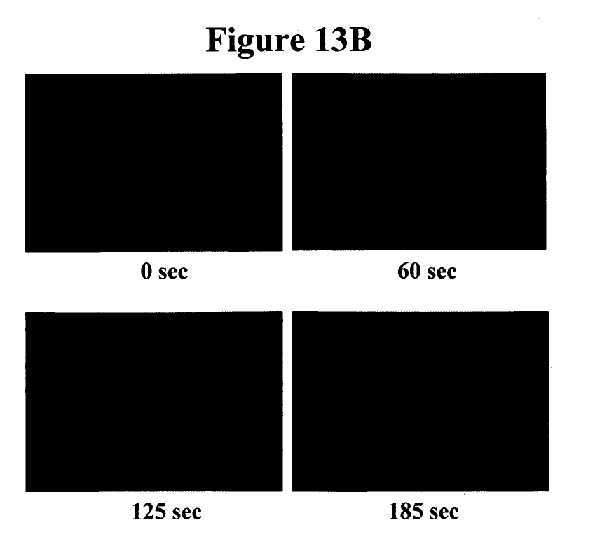

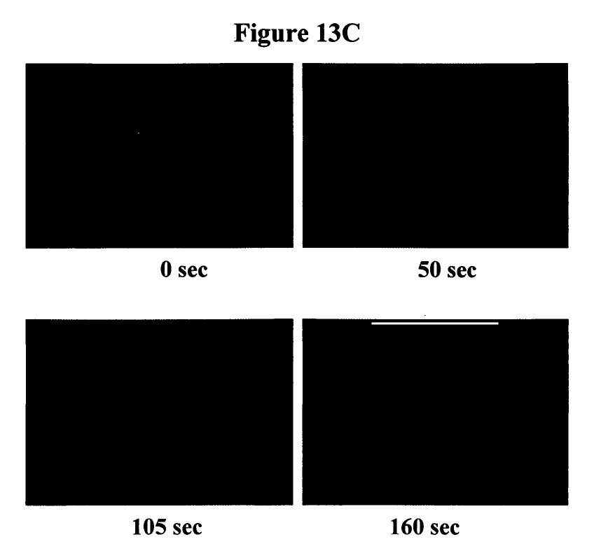

FIG. 13 depicts the fluidity of the generated lipid bilayer. In FIG. 13A, lipid bilayers comprising POPC and DOPE-NDB were photobleached over an initial area with a diameter of 30 μm. The fluidity of the lipid bilayer is seen in the recovery of the photobleached area within 30 seconds. Additionally, a lipid bilayer comprising POPC/DOPE-bio and bound by SA-FITC in DPBS with 1 mg/ml BSA was capable of recovering from photobleaching on a similar time scale (FIG. 13B). Similar results were also seen with a POPC/DOPE-1% NTA-Ni bilayer bound with 6×His tagged ICAM-1-FITC (FITC-conjugates intracellular adhesion molecule-1) (FIG. 13C).

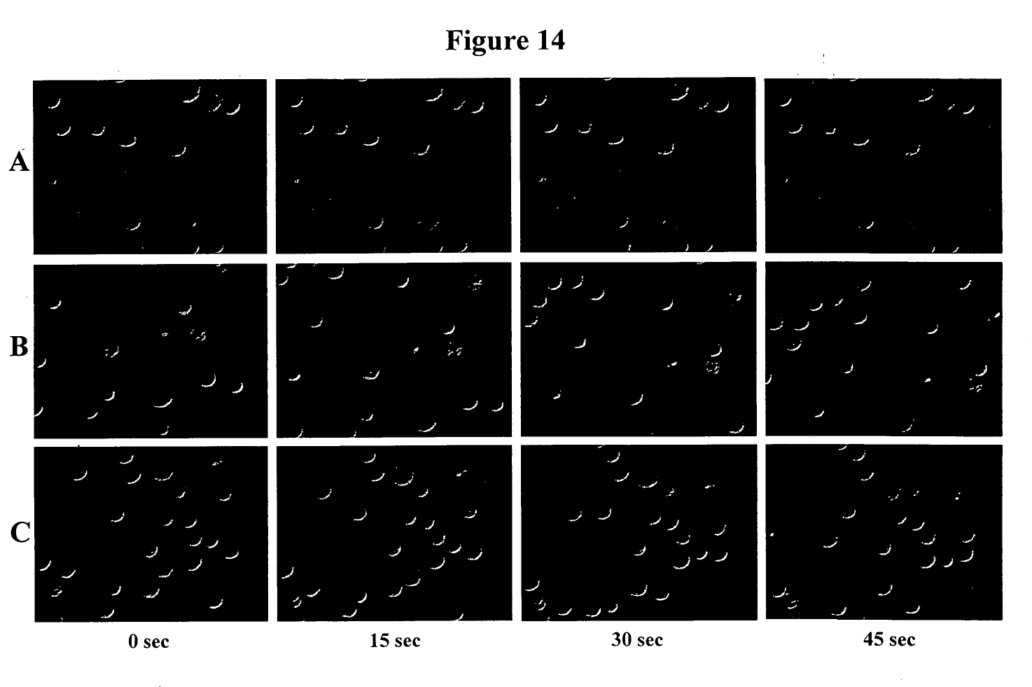

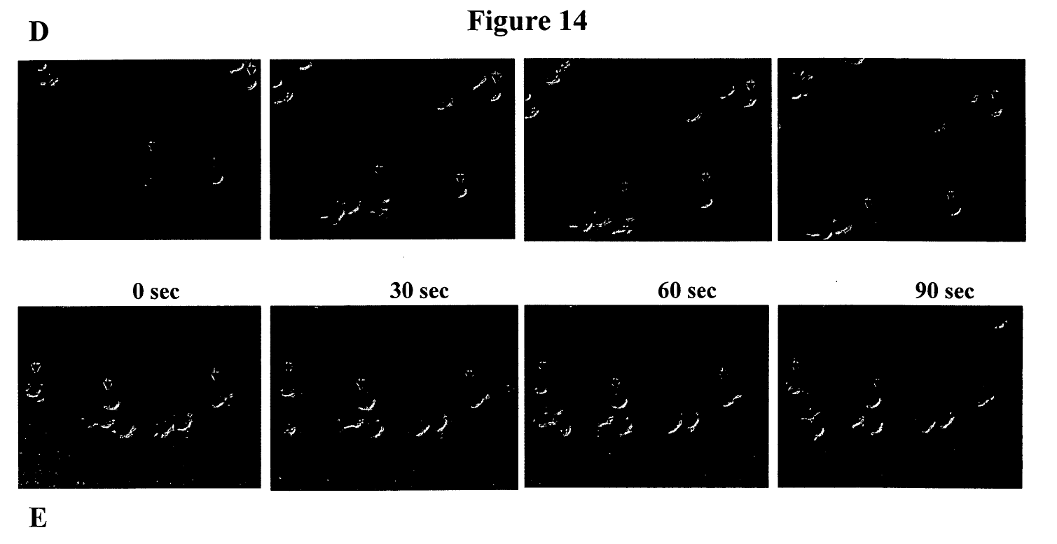

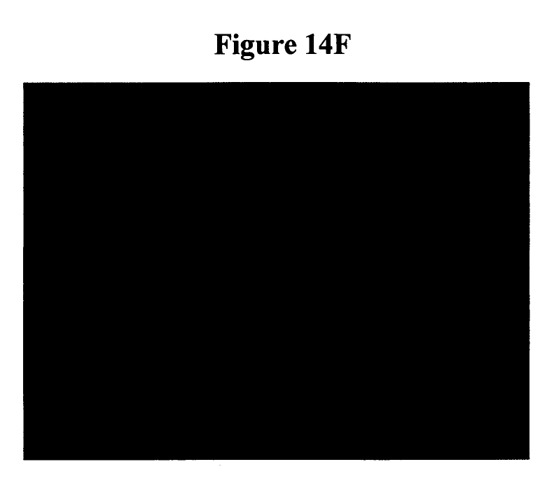

The ability of cells to move freely over various lipid bilayers was assayed. In FIG. 14A, D10 T cells are shown to be adhered to glass (see arrows). Cells were freely mobile on glass covered by a lipid bilayer comprising POPC/1% DOPE-NBD/1% DOPE-bio (FIG. 14B). Cells on lipid bilayers comprising POPC/1% DOPE-NBD/1% DOPE-NTA-Ni were also freely mobile, though not as mobile as on lipid bilayer comprising POPC/1% DOPE-NBD/1% DOPE-bio, due probably, in part, to the charge on NTA-Ni (FIG. 14C). The ability to anchor cells to POPC/1% DOPE-NBD/0.5% DOPE-bio lipid bilayers was subsequently tested with phorbol 12-myristate 13-acetate (PMA) activated D10 cells. In the absence of ligand, cells PMA activated cells were still mobile over the lipid bilayer in HSPG pH 7.4, 2 mM MgCl₂, 1 mM CaCl₂, 10% BSA, 50 ng/ml PMA (FIG. 14D). However, when SA-(ICAM-1)₂ was added to the lipid bilayer first, the activated D10 cells adhered to the lipid bilayer (FIG. 14E). Notably, the adherence of the D10 T cells did not create any visual defects in the lipid bilayer (FIG. 14F; buffer: HSPG pH 7.4, 5 mM MgCl₂, 0.5 mM MnCl₂, 50 μM CaCl₂, 4% FBS).

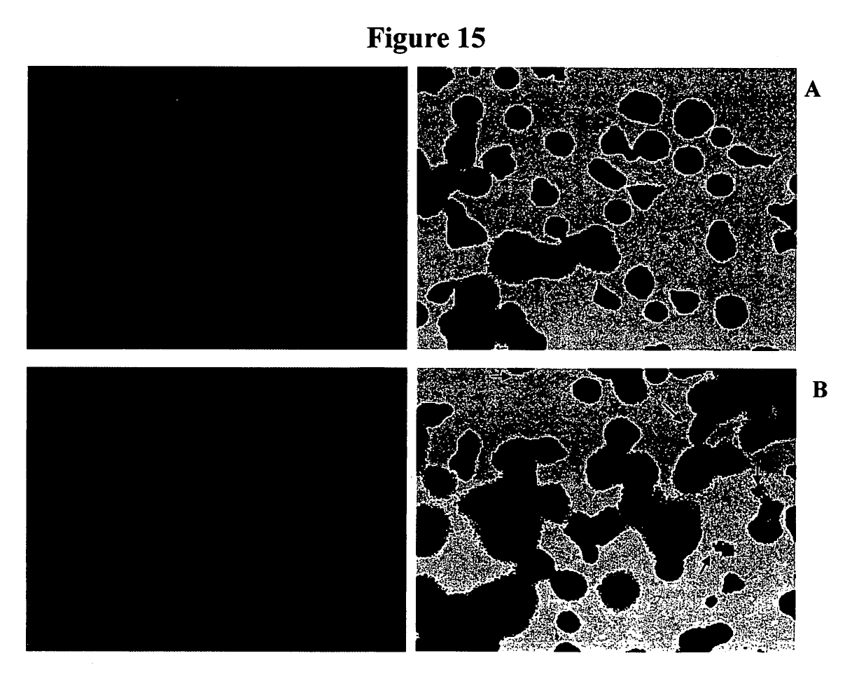

Additionally, the ability of the lipid bilayer to bind ligands and subsequently activate T cells was tested. PMA stimulated D10 T cells were contacted with lipid bilayers comprising POPC/1% DOPE-NBD/1% DOPE-bio in HSPG pH 7.4, 5 mM MgCl₂, 0.5 mM MnCl₂, 50 μM CaC₂, 4% FBS bound with SA-(ICAM-1)₂ (FIG. 15A) or with SA-(ICAM-1)₂ and SA-(I-Ek-MCC)₂ (FIG. 15B). As seen by the calcium flux studies in FIG. 15, T cells were activated only in the presence of I-Ek multimers.

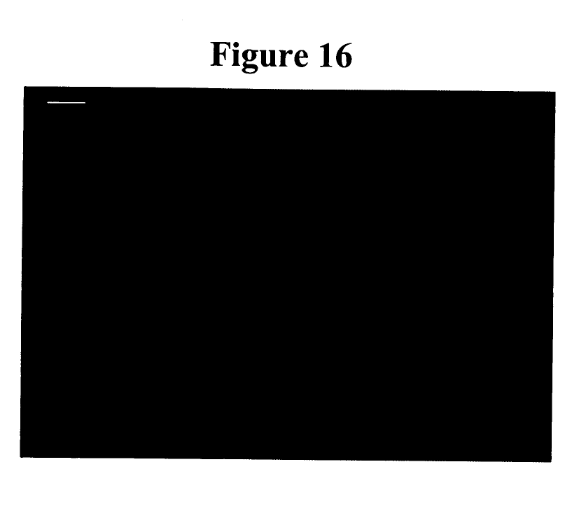

As seen in FIG. 16, the leading front of a POPC/1% DOPE-NBD/1% DOPE-bio lipid bilayer is capable of binding streptavidin-FITC in DPBS, 1 mg/ml BSA, pH 7.4.

The artificial cell membrane system described herein closely mimics the cell surface and provides a valuable research tool to study the subtle differences in intracellular signaling pathways induced by specific ligand/receptor or membrane/membrane interactions. These differences provide potential targets for intervention by therapeutics. It is known that many viruses, for example, enter host cells through membrane fusion mediated by specific interactions between cellular receptors and viral envelope proteins. The artificial membrane system of the present invention can be used to study the mechanism of viral entry and the effects of agents which block this process. Cell-cell contact or cell-extracellular matrix contact in other systems, such as those which occur between components of the nervous system, can also be studied employing the artificial membrane system disclosed herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for producing a fluid planar lipid layer-based membrane-anchored ligand system with defined ligand valency comprising:
a) contacting a solid surface with a lipid layer containing lipids conjugated to a first specific binding pair member;
b) functionally linking a ligand to a second specific binding pair member which has binding affinity for said first binding pair member, said second member comprising at least one binding site for binding said first member; and
c) contacting the lipid layer of step a) with the linked ligand of step b) whereby contact of the lipid layer with said second binding pair member functionally linked to said ligand results in anchoring of the ligand to said lipid, thereby forming a fluid planar lipid layer-based membrane-anchored ligand system with defined ligand valency.

2. The method of claim 1, wherein said ligand is functionally linked to said second binding pair member through binding interaction with said first binding pair member.

3. The method of claim 1 further comprising at least one cell comprising a receptor having binding affinity for said at least one ligand.

4. The method of claim 1 further comprising a virus comprising a receptor having binding affinity for said at least one ligand.

5. A fluid planar lipid layer-based membrane-anchored ligand system produced by the method of claim 1.

6. The fluid planar lipid layer-based membrane-anchored ligand system of claim 4, wherein said at least one ligand is selected from the group consisting of I-EK-MCC and I-AK-CA, neuropilin-1, LFA1, DC-SIGN, ICAM1, ICAM3, MHC, TCR, CD100, SEMA4A, CD40, CD40L, CD80, CD86, CD28, SEMA7A, CD72, TIM2, B7-H1/B7-DC, B7-1/B7-2, B7RP-1, B7H3, 4-1BBL, CD27L, OX40L, OX40, CD27, 4-1BB, ICOS, CTLA4, PD1, plexin-C1, CD4, CD8, CKR family members, CXCR4, CCR5, CCR3, gamma-cytokine receptor family members, IL2R, IL4R, IL7R, IL15R, SRA, CD68, LOX1, HSP receptors, CD91, TLR4, TLR2, CD36, CD40, CD14, v3 integrin, and TNFR family members, TNFR, FAS, and FASL.

7. The method of claim 1, wherein said surface is selected from the group consisting of a glass coverslip, a biacore chip, a sensor chip or a tissue culture plate.

8. The method of claim 1, wherein said first binding pair member is biotin and said second binding pair member comprises a plurality of binding sites for said first member and is selected from the group consisting of streptavidin or avidin.

9. The method of claim 1, wherein said first binding pair member is nickel and said second binding pair member is a histidine tag.

10. The method of claim 1, wherein said specific binding member pairs are selected from the group consisting of nickel-histidine, biotin-streptavidin, antibody-antigen, lectin-carbohydrate, and complementary oligonucleotides.

11. The method of claim 3, wherein said at least one cell is selected from the group consisting of a T cell, an antigen presenting cell, a macrophage, a B cell, a neuron, a fibroblast, an endothelial cell, an epithelial cell, a synoviocyte, a muscle cell, a stem cell, and a dendritic cell.

12. The method of claim 4, wherein said virus is HIV.

13. The method of claim 1, wherein said lipids are selected from the group consisting of POPC, DOPC, and derivatives thereof.

14. A kit for practicing the method of claim 1, comprising:
a) lipids;
b) a solid surface;
c) a plurality of first and second binding members; and
d) optionally at least one ligand of interest.

15. The kit of claim 14, further comprising viable cells, appropriate buffers, gel filtration apparatus, detectable labels and instructional material.
